(19) United States
(12) Patent Application Publication
(10) Pub. No.: US 2013/0217045 A1
(43) Pub. Date: Aug. 22, 2013

(54) Use of Functional Autoantibodies in Alzheimer Disease

(71) Applicants: Jun Tan, Tampa, FL (US); Demian Obregon, Tampa, FL (US); Huayan Hou, Tampa, FL (US); Juan Deng, Tampa, FL (US)

(72) Inventors: Jun Tan, Tampa, FL (US); Demian Obregon, Tampa, FL (US); Huayan Hou, Tampa, FL (US); Juan Deng, Tampa, FL (US)

(73) Assignee: University of South Florida (A Florida Non-Profit Corporation), Tampa, FL (US)

(21) Appl. No.: 13/768,158

(22) Filed: Feb. 15, 2013

Related U.S. Application Data
(60) Provisional application No. 61/600,313, filed on Feb. 17, 2012.

(51) Int. Cl. G01N 33/68

(52) U.S. Cl. CPC G01N 33/6854
USPC 435/7.92; 435/7.1

(57) ABSTRACT

Provided herein is a method for diagnosing Alzheimer's disease in a subject comprising detecting an increase in an amyloidogenic Aβ₁₋₁₇ antibody in the subject as compared to a control. Further provided herein is a method for testing efficacy of an Alzheimer's disease treatment in a subject comprising detecting a decrease in an amyloidogenic Aβ₁₋₁₇ antibody in the subject as compared to prior to the treatment.

### CROSS REFERENCE TO RELATED APPLICATIONS

This application claims the priority benefit of U.S. Provisional Patent Application Ser. No. 61/600,313 filed on Feb. 17, 2012, in the United States Patent Office.

### STATEMENT REGARDING FEDERALLY SPONSORED RESEARCH OR DEVELOPMENT

This invention was made with Government support under Contract No. R01AG032432 and R42AG031586 awarded by the NIH/NIA and a Veterans Affairs Merit grant (JT). The U.S. Government has certain rights in this invention.

### BACKGROUND OF THE INVENTION

1) Field of the Invention

The present invention relates to the field of immunology and Alzheimer's disease.

2) DESCRIPTION OF RELATED ART

Alzheimer's disease (AD) is a neurodegenerative disorder and the most common cause of dementia. In the brains of AD patients, amyloid-β (Aβ) peptides, derived from the amyloid precursor protein (APP), accumulate into b-amyloid plaques, one of the pathologic hallmarks of the disease. Neurotoxic oligomeric forms of Aβ are hypothesized to play a critical role in AD pathogenesis [Walsh, D. M., Klyubin, I., Fadeeva et al. (2002) Nature, 416(6880), 535-539; Lesné, S., Koh, M. T., Kotilinek, L. et al. (2006) Nature, 440(7082), 352-357; Haass, C. & Selkoe, D. J. (2007) Nature Reviews Molecular Cell Biology, 8(2), 101-112]. Previous studies suggest that both endogenous naturally occurring anti-Aβ autoantibodies, or those generated by vaccination against Aβ, may enhance clearance of the peptide from the brain [Schenk, D., Barbour R., Dunn W. et al. (1999) Nature Cell Biology 6, 1054-1061; Morgan D., Diamond, D. M., Gottschall, P. E. et al. (2000) Nature 408, 982-985; Dodel, R. C., Du Y., Depboylu, C. et al. (2004) Proc. National Academy of Science USA 98, 8850-8855; Morgan D. (2011) Journal of Internal Medicine 269, 54-63].

Indeed, active or passive immunization against Aβ peptide has been proposed as a method for preventing and treating AD [Schenk, D., Barbour R., Dunn W. et al. (1999) Nature Cell Biology 6, 1054-1061; Morgan D. (2011) Journal of Internal Medicine 269, 54-63]. Active immunization in transgenic AD mice reduced fibril formation, enhanced clearance of Aβ plaques, and improved behavioral impairment [Schenk, D., Barbour R., Dunn W. et al. (1999) Nature Cell Biology 6, 1054-1061; Morgan D., Diamond, D. M., Gottschall, P. E. et al. (2000) Nature 408, 982-985; Morgan D. (2011) Journal of Internal Medicine 269, 54-63]. In addition, passive immunization with antibodies recognizing the N-terminal and central domains of Aβ peptides was also effective [DeMattos, R. B., Bales, K. R., Cummins, D. J. et al. (2001) Proc. National Academy of Science USA 98, 8850-8855]. In patients vaccinated against the N-terminus of Aβ, considerable decreases in plaque load have been reported, but this clearance of pre-formed plaques was not sufficient to improve cognitive function in AD patients [Holmes, C., Boche, D., Wilkinson, D. et al. (2008). The Lancet, 372(9634), 216-223]. Similarly, passive vaccination of transgenic AD mice against the N-terminus of Aβ inhibited fibril formation and disaggregated pre-formed amyloid fibrils; however, it did not disrupt toxic oligomers [Mamikonyan, G., Necula, M., Mkrtichyan, M. et al. (2007). The Journal of Biological Chemistry 282(31), 22376-22386].

Notably, the first AD vaccine AN1792, was based on a synthetic form of Aβ₁₋₄₂. In phase II trials (N=372 with mild to moderate AD), about 6% of patients developed meningoencephalitis and leukoencephalopathy, causing the trial to be halted [Orgogozo, J. M., Gilman, S., Dartigues, J. F. et al. (2003) Neurology, 61(1), 46-54]. Importantly in that study, immunization resulted in generation of anti-Aβ antibodies targeting the N-terminal Aβ. However, previous studies suggested that it is the Aβ₁₅₋₄₂ region which initiated T-cell responses that triggered the meningoencephalitis. The B-cell epitope Aβ₁₁₋₁₅ is considered to be important for generation of anti-Aβ antibodies [Monsonego, A., Zota, V., Karni, A. et al. (2003) Journal of Clinical Investigation 112(3), 415-422; Lee, M., Bard, F., Johnson-Wood, K. et al. (2005) Annals of Neurology 58, 430-435; Pride, M., Seubert, P., Grundman, M. et al. (2008) Neurodegenerative Diseases 5(3-4), 194-196].

A number of past studies have quantified autoantibodies against Aβ in AD. Some investigators found reduced anti-Aβ autoantibodies in AD patients [Du, Y., Dodel, R., Hampel, H. et al. (2001) Neurology, 57(5), 801-805; Weksler, M. E., Relkin, N., Turkenich, R. et al. (2002) Experimental Gerontology, 37(7), 943-948] compared with controls. However, a more recent study indicates that such autoantibodies against the most toxic species of Aβ are reduced in both normal elderly and AD patients [Britschgia, M., Olina, C. E., Johnsa, H. T., et al. (2009) Proc. National Academy of Science USA 106, 12145-12150]. Anti-Aβ autoantibodies are generally believed to promote clearance of the peptide from the brain [Dodel, R. C., Du Y., Depboylu, C. et al. (2004) Proc. National Academy of Science USA 98, 8850-8855; Taguchi, H., Planque, S., Nishiyama, Y. et al. (2008) Journal of Biological Chemistry 283(8), 4714-4722; Bacher, M., Depboylu, C., Du, Y. et al. (2009) Neuroscience Letters 449(3), 240-245]. Indeed, natural autoantibodies comprise some two-thirds of the total adult human antibody pool and are multifunctional [Shoenfeld, Y., Cervera, R., Haass, M. et al. (2007) Annals of the New York Academy of Sciences 1109, 138-144]. While the concentrations and binding of anti-Aβ antibodies to Aβ have been extensively studied, knowledge of their functional effects on APP processing is unknown.

### BRIEF DESCRIPTION OF THE DRAWINGS

FIG. 1 shows that concentrated Aβ autoantibodies from AD patients promote β-secretase cleavage of APP in CHO/APPswe/PS1wt cells. FIG. 1 includes a dot plot (a), immunoblot (b) and bar graph (c).

FIG. 2 shows that treatment with Aβ antibody against N-terminal 1-17 peptide (6E10) increases Aβ production in cultured cells. FIG. 2 includes immunoblots (a-e) and fluorescent microscopy images (f-g).

FIG. 3 shows that treatment with Aβ₁₋₁₇ antibody dose-dependently increases Aβ production. FIG. 3 includes bar graphs (a, d) and immunoblots (b, c, e, and f).

FIG. 4 shows that treatment with Aβ₁₋₁₇ antibody promotes APP β-secretase cleavage. FIG. 4 provides three immunoblots (a-c).

FIG. 5 shows that Aβ₁₋₁₇ antibody modulates APP processing in vivo. FIG. 5 provides two immunoblots (a-b).

### DETAILED DESCRIPTION OF THE INVENTION

Provided herein is a method for diagnosing Alzheimer's disease in a subject comprising detecting an increase in an amyloidogenic Aβ₁₋₁₇ antibody in the subject as compared to a control. Also provided herein is a method for prognosing an Alzheimer's disease in a subject comprising detecting an increase or a decrease in an amyloidogenic Aβ₁₋₁₇ antibody in the subject as compared to a control, wherein an increase indicates a poor prognosis and a decrease indicates a more favorable prognosis. Further provided herein is a method for testing efficacy of an Alzheimer's disease treatment in a subject comprising detecting a decrease in an amyloidogenic Aβ₁₋₁₇ antibody in the subject as compared to prior to the treatment.

Terms used throughout this application are to be construed with ordinary and typical meaning to those of ordinary skill in the art. However, Applicants desire that the following terms be given the particular definition as defined below.

### DEFINITIONS

As used in the specification and claims, the singular form “a,” “an” and “the” includes plural references unless the context clearly dictates otherwise. For example, the term “a cell” includes a plurality of cells, including mixtures thereof.

The term “Alzheimer's disease” is defined herein as a form of dementia or cognitive disfunction. The term “Alzheimer's disease” includes each stage of the condition (mild, moderate and severe Alzheimer's disease). Alzheimer's disease includes, but is not limited to, one or more of the following conditions: difficulty remembering recent events; memory loss that occurs with regularity; organizational difficulties; poor judgment; confusion; irritability; aggression; mood swings; trouble with language; inability to perform complex tasks; inability to recognize family members and/or friends; long-term memory loss; difficulty following instructions; difficulty sleeping at night; having hallucinations, delusions, paranoia, or compulsive behaviors; inability to walk, talk and care of oneself; difficulty eating; and difficulty controlling urinations and bowel movements.

The term “amyloid precursor protein” (APP) refers to a polypeptide that is encoded by an APP gene as described in the HUGO Gene Nomenclature Committee Database under HGNC ID No. 620. Amyloid precursor proteins are cleaved by secretase enzymes in vivo, which cleavage produces APP fragments sAPP-α, sAPP-β, and Aβ. Cleavage of APP by α-secretase results in two APP fragments: sAPP-α and CTF-α. Since α-secretase cleaves APP close to the transmembrane region of the APP protein, sAPP-α contains much of the extracellular domain of APP. CTF-α contains the remainder of the APP polypeptide, a C-terminal fragment (CTF), following cleavage by α-secretase. Cleavage of APP by β-secretase results in two APP fragments: sAPP-β and CTF-β. Since 13-secretase also cleaves APP close to the transmembrane region of the APP protein, sAPP-β contains much of the extracellular domain of APP. CTF-β contains the remaining C-terminal portion of APP following cleavage by β-secretase. An Aβ fragment is created by cleavage of APP by β-secretase followed by cleavage of CTF-β by γ-secretase. Accordingly, a CTF-β fragment contains an Aβ amino acid sequence and can be identified using an antibody to an Aβ fragment. Each APP fragment can be identified by commercially available antibodies (some of which are described below) and methods known to those of ordinary skill in the art.

The term “amyloidogenic Aβ₁₋₁₇ antibody” refers to an antibody that 1) binds to a region of a beta amyloid (Aβ) polypeptide including all or a portion of amino acids 1-17 and 2) increases amyloid precursor protein (APP) amyloidogenic processing. An increase in APP amyloidogenic processing can be indicated by 1) an increase in a sAPP-β as compared to a control, 2) a decrease in a sAPP-α as compared to a control, and/or 3) an increase in the ratio of a β-CTF to an α-CTF as compared to a control.

The term “amyloidogenic” refers herein to a process or a compound that is likely to, or does, generate an amyloid.

The term “antibody” is used in the broadest sense, and specifically covers monoclonal antibodies (including full-length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such a molecule from having the ability to bind to the high affinity receptor, FcεRI. As used herein, “functional fragment” with respect to antibodies refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind to a target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains, which enables the sFv to form the desired structure for target binding.

The term “APP cleavage assay” refers herein to any assay that detects one or more cleavage products of APP (or APP fragments) including, but not limited to, sAPP-β, sAPP-α, β-CTF, α-CTF and Aβ.

The term “beta amyloid” (Aβ) refers to a polypeptide of approximately 36-49 or 39-42 amino acids that is derived from or situated within an APP. The term beta amyloid includes a polypeptide that consists of 40 amino acids (Aβ₄₀) and a polypeptide that consists of 42 amino acids (Aβ₄₂). Due to its hydrophobic nature, an Aβ₄₂ polypeptide tends to be more amyloidogenic. It should be understood that an antibody that binds to all or a portion of amino acids 1-17 of Aβ can bind to either or both the Aβ polypeptide that is derived from an APP and the Aβ polypeptide as it is situated within an APP sequence prior to secretase cleavage of the APP.

The terms “cell,” “cell line” and “cell culture” include progeny. It is also understood that all progeny may not be precisely identical in DNA content due to deliberate or inadvertent mutations. Variant progeny that have the same function or biological property, as screened for in the originally transformed cell, are included. The “host cells” used in the present invention generally are prokaryotic or eukaryotic hosts.

A “composition” is intended to mean a combination of active agent and another compound or composition, inert (for example, a detectable agent or label) or active, such as an adjuvant.

As used herein, the term “comprising” is intended to mean that the compositions and methods include the recited elements but do not exclude others. “Consisting essentially of,” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purpose. A control can be “positive” or “negative.” In some embodiments, a control is a sample obtained from a healthy subject. In other embodiments, a control is a sample obtained from a subject prior to treatment of the subject or prior to a given treatment of the subject. In still other embodiments, a control is a sample containing β-actin. In these embodiments, an increase or decrease in an APP cleavage product can be expressed as a ration of the cleavage product to β-actin.

“Differentially expressed” as applied to a gene refers to the differential production of the mRNA transcribed from the gene or the protein product encoded by the gene. A differentially expressed gene may be overexpressed or underexpressed as compared to the expression level of a normal or control cell. In one aspect, it refers to a differential that is 2.5 times, preferably 5 times, or preferably 10 times higher or lower than the expression level detected in a control sample. The term “differentially expressed” also refers to nucleotide sequences in a cell or tissue which are expressed in a sample cell and silent in a control cell or not expressed in a sample cell and expressed in a control cell.

An “effective amount” is an amount sufficient to effect beneficial or desired results. An effective amount can be administered in one or more administrations, applications or dosages.

As used herein, “expression” refers to the process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. “Overexpression” as applied to a gene refers to the overproduction of the mRNA transcribed from the gene or the protein product encoded by the gene at a level that is 2.5 times higher, preferably 5 times higher, more preferably 10 times higher, than the expression level detected in a control sample.

The Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

A “gene” refers to a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art.

A “gene product” refers to the amino acid (e.g., peptide or polypeptide) generated when a gene is transcribed and translated.

“Humanized” forms of non-human (e.g. murine) antibodies are chimeric immunoglobulins, immunoglobulin chains or fragments thereof (such as Fv, Fab, Fab′, F(ab′)₂ or other target-binding subsequences of antibodies) which contain minimal sequence derived from non-human immunoglobulin. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc).

The term “identity” or “homology” shall be construed to mean the percentage of amino acid residues in the candidate sequence that are identical with the residue of a corresponding sequence to which it is compared, after aligning the sequences and introducing gaps, if necessary, to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software.

The term “isolated” means separated from constituents, cellular and otherwise, in which the polynucleotide, peptide, polypeptide, protein, antibody, or fragments thereof, are normally associated with in nature. In one aspect of this invention, an isolated polynucleotide is separated from the 3′ and 5′ contiguous nucleotides with which it is normally associated with in its native or natural environment, e.g., on the chromosome. As is apparent to those of skill in the art, a non-naturally occurring polynucleotide, peptide, polypeptide, protein, antibody, or fragments thereof does not require “isolation” to distinguish it from its naturally occurring counterpart. In addition, a “concentrated,” “separated” or “diluted” polynucleotide, peptide, polypeptide, protein, antibody, or fragments thereof is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

The word “label” when used herein refers to a detectable compound or composition which can be conjugated directly or indirectly to a molecule or protein, e.g., an antibody. The label may itself be detectable (e.g., radioisotope labels or fluorescent labels) or, in the case of an enzymatic label, may catalyze chemical alteration of a substrate compound or composition which is detectable.

“Mammal” for purposes of treatment refers to any animal classified as a mammal, including humans, domestic and farm animals, nonhuman primates, and zoo, sports, or pet animals, such as dogs, horses, cats, cows, etc.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single target site. Furthermore, in contrast to conventional (polyclonal) antibody preparations, which typically include different antibodies directed against different determinants (epitopes), each monoclonal antibody is directed against a single determinant on the target. In addition to their specificity, monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies for use with the present invention may be isolated from phage antibody libraries using the well-known techniques. The parent monoclonal antibodies to be used in accordance with the present invention may be made by the hybridoma method first described by Kohler and Milstein (Nature 256, 495 (1975)) or may be made by recombinant methods.

The terms “polynucleotide” and “oligonucleotide” are used interchangeably and refer to a polymeric form of nucleotides of any length, either deoxyribonucleotides or ribonucleotides or analogs thereof. Polynucleotides may have any three-dimensional structure and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine (T) when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

A “subject,” “individual” or “patient,” used interchangeably herein, refers to a vertebrate, preferably a mammal, more preferably a human. Mammals include, but are not limited to, murines, simians, humans, farm animals, sport animals, and pets.

The phrase “substantially identical” with respect to an antibody chain polypeptide sequence may be construed as an antibody chain exhibiting at least 70%, or 80%, or 90%, or 95% sequence identity to the reference polypeptide sequence. The term with respect to a nucleic acid sequence may be construed as a sequence of nucleotides exhibiting at least about 85%, or 90%, or 95%, or 97% sequence identity to the reference nucleic acid sequence.

The terms “treat,” “treating,” “treatment” and grammatical variations thereof as used herein include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Nevertheless, it should be understood that an “Alzheimer's disease treatment” is considered a “treatment” upon administration to a subject and that such treatment does not require efficacy in the subject. Provided herein are methods for determining the efficacy of such treatment.

The term “variable” in the context of variable domain of antibodies refers to the fact that certain portions of the variable domains differ extensively in sequence among antibodies and are used in the binding and specificity of each particular antibody for its particular target. However, the variability is not evenly distributed through the variable domains of antibodies. It is concentrated in three segments called complementarity determining regions (CDRs) also known as hypervariable regions both in the light chain and the heavy chain variable domains. The more highly conserved portions of variable domains are called the framework (FR). The variable domains of native heavy and light chains each comprise four FR regions, largely by adopting a .beta.-sheet configuration, connected by three CDRs, which form loops connecting, and in some cases forming part of, the .beta.-sheet structure. The CDRs in each chain are held together in close proximity by the FR regions and, with the CDRs from the other chain, contribute to the formation of the target binding site of antibodies (see Kabat et al., Sequences of Proteins of Immunological Interest, National Institute of Health, Bethesda, Md. 1987). As used herein, numbering of immunoglobulin amino acid residues is done according to the immunoglobulin amino acid residue numbering system of Kabat et al. (Sequences of Proteins of Immunological Interest, National Institute of Health, Bethesda, Md. 1987), unless otherwise indicated.

### Compositions and Methods

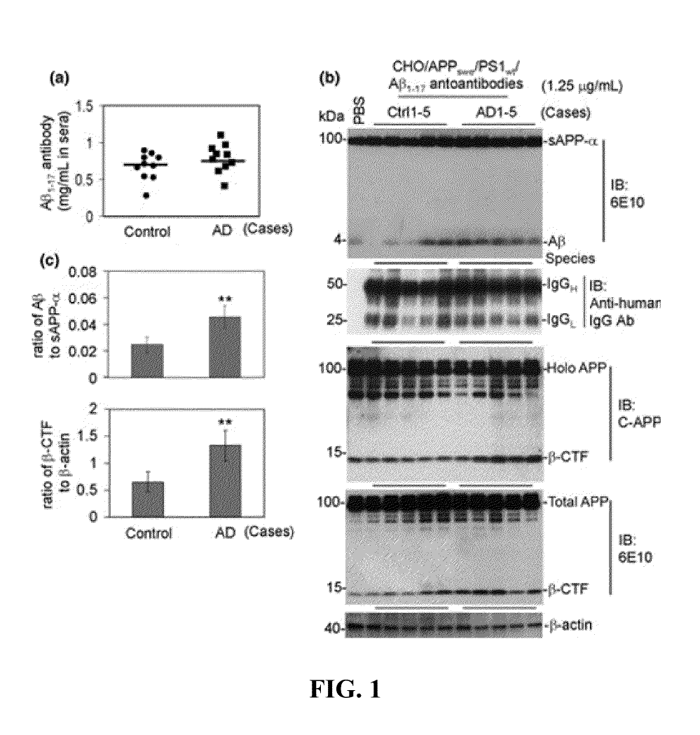

Provided herein is a method for diagnosing Alzheimer's disease in a subject comprising detecting an increase in an amyloidogenic Aβ₁₋₁₇ antibody in the subject as compared to a control. A surprising discovery provided herein is that AD patients have an increase in naturally occurring concentrated autoantibodies that actually promote amyloidogenic processing of APP as compared with non-demented controls (FIG. 1). Amyloidogenic processing of APP results in an increase in Aβ species such as Aβ₄₀ and Aβ₄₂ that have been implicated in amyloid plaque formation and the development of Alzheimer's disease. Accordingly, detecting these amyloidogenic Aβ₁₋₁₇ antibodies provides a means to diagnose Alzheimer's disease.

This discovery is quite surprising in that it seems to be contrary to the teachings of the prior art which describe that anti-Aβ autoantibodies promote clearance of the deleterious Aβ peptide from the brain [Dodel, R. C., Du Y., Depboylu, C. et al. (2004) Proc. National Academy of Science USA 98, 8850-8855; Taguchi, H., Planque, S., Nishiyama, Y. et al. (2008) Journal of Biological Chemistry 283(8), 4714-4722; Bacher, M., Depboylu, C., Du, Y. et al. (2009) Neuroscience Letters 449(3), 240-245]. In contrast, the present findings suggest that some, or certain subsets, of autoreactive Aβ antibodies may indeed be deleterious, rather than salutary due to their previously reported amyloid-clearing capability.

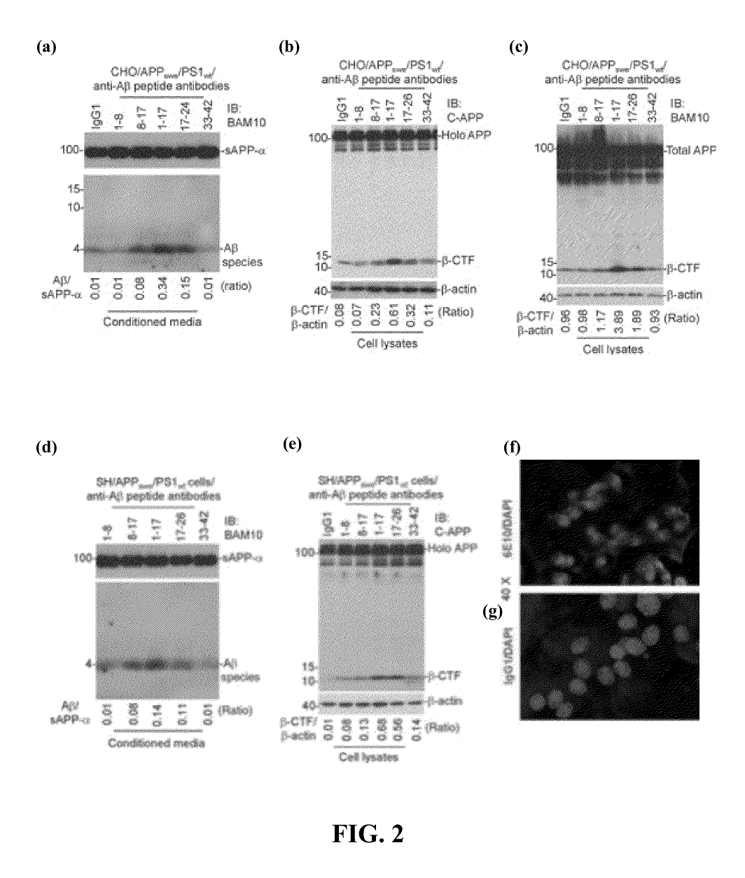

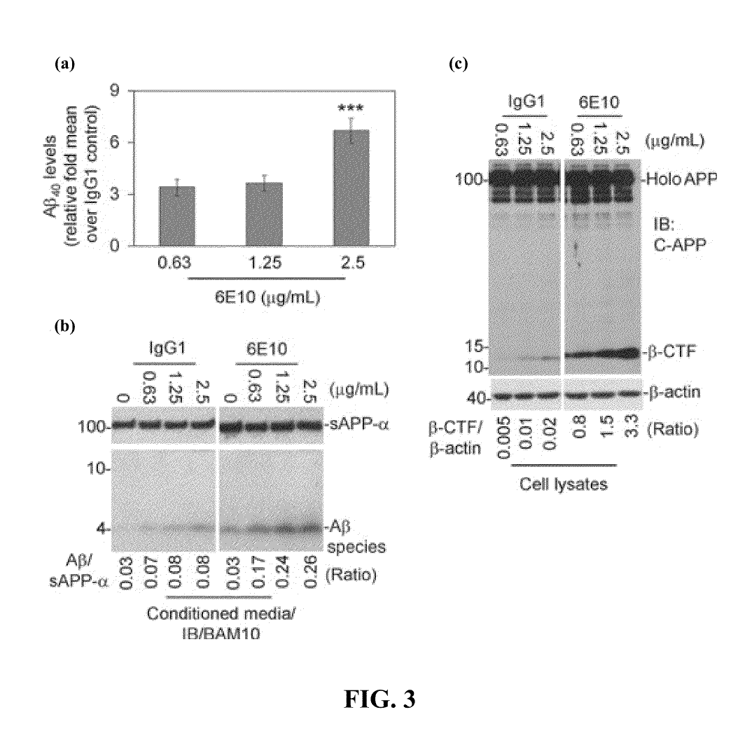

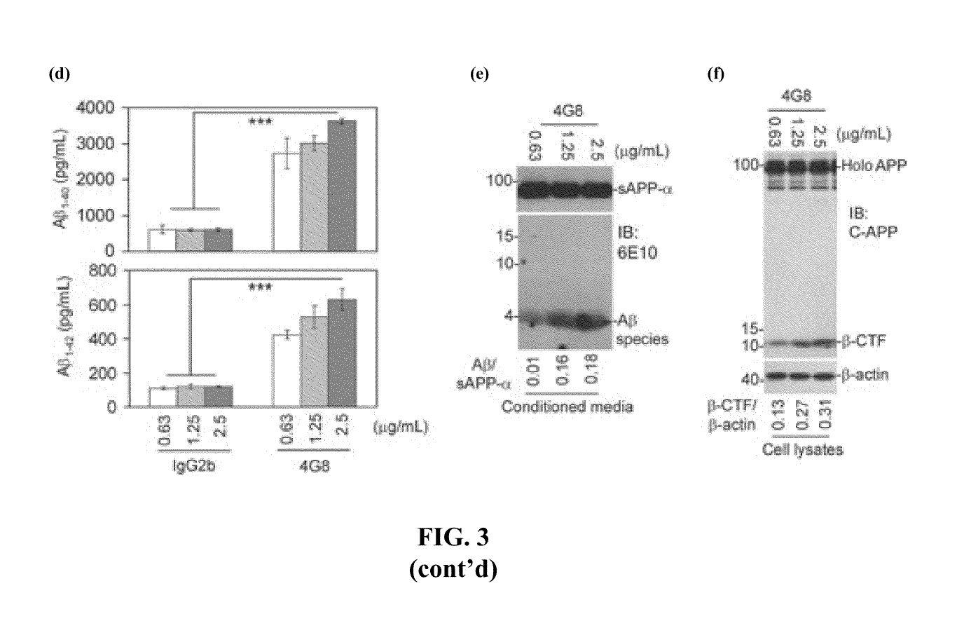

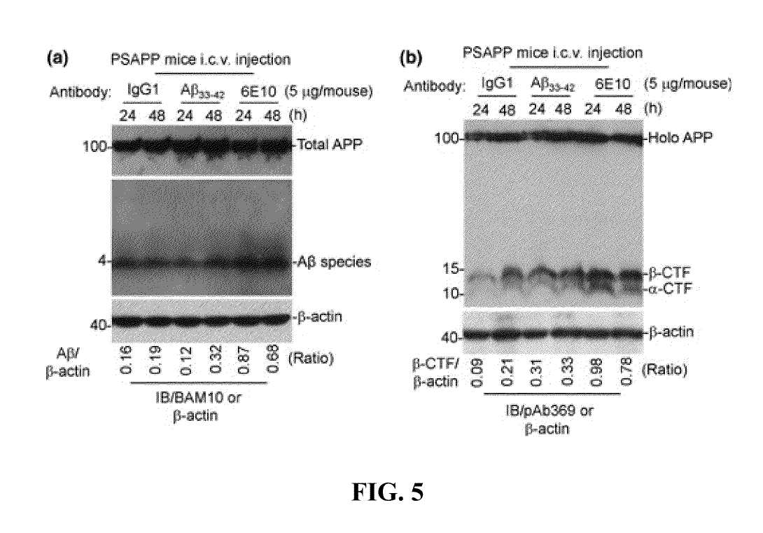

The data provided herein suggests that the most potent autoantibodies from AD serum for promoting Aβ generation are those targeting the N-terminal extracellular region of Aβ, specifically Aβ₁₋₁₇, as antibodies against this region increased indicators of β-secretase processing of APP, specifically total Aβ and β-CTF (FIG. 2). To determine whether the increase in β-secretase activity observed in the AD clinical population (FIG. 1) could be modeled in vivo, PSAPP mice were treated at 8 months of age with i.c.v. Aβ₁₋₁₇ antibody, Aβ₃₃₋₄₂ antibody, or IgG1 control at 5 μg/mouse; based on the upper limits of Aβ₁₋₁₇ patient blood (FIG. 1) and in vitro (FIGS. 2 and 3) studies. It was determined that addition of the anti-Aβ₁₋₁₇ antibody (6E10) significantly increased Aβ production (FIG. 5a) compared with the Aβ₃₃₋₄₂ antibody or IgG1 control in these mice.

In addition to increasing β-secretase activity, the anti-N-terminal Aβ antibody (6E10) against Aβ₁₋₁₇ peptide also appeared to dose-dependently promote amyloidogenic processing of APP via blockade of α-secretase APP cleavage. Importantly, the corresponding Aβ₁₋₁₇ region of APP contains the α-secretase cleavage site. Therefore, α-secretase activity may putatively be competitively blocked by Aβ₁₋₁₇ antibody binding. Additionally, the ratio of β- to α-CTF was significantly higher by immunoblot analysis which was another indicator of amyloidogenic APP processing by β-secretase being associated with anti-Aβ₁₋₁₇ antibody (FIG. 5b).

Soluble Aβ species, including Aβ₄₂ and resulting multimeric aggregates, have been shown recently in vitro and in transgenic mice models to be crucial toxic species [Cleary, J. P., Walsh, D. M., Hofineister, J. J. et al. (2005) Nature Neuroscience 8(1), 79-84; Klyubin, I., Walsh, D. M., Lemere, C. A. et al. (2005) Nature Medicine 11(5), 556-561; Lesné, S., Koh, M. T., Kotilinek, L. et al. (2006) Nature 440(7082), 352-357; Townsend, M., Shankar, G. M., Mehta, T. et al. (2006) Journal of Physiology 572(2), 477-492; Glabe, C. G. (2008) Journal of Biological Chemistry 283(44), 29639-29643; Shanker, G. M., Li, S., Mehta, T. H. et al. (2008) Nature Medicine 14, 837-842; Tomic, J. L., Pensalfini A., Head, E., and Glabe, C. G. (2009) Neurobiology of Disease 35, 352-358]. Furthermore, small Aβ oligomers may form intracellularly before being released into the extracellular medium, where they can interfere with synaptic activity or act as seeds to promote fibrillization [Selkoe, D. J. (2004) Nature Cell Biology 6(11), 1054-1061; Khandogin, J. & Brooks, C. L. (2007) Proc. National Academy of Science 104(43), 16880-16885]. The data provided herein indicates that autoantibodies such as those directed to the Aβ₁₋₁₇ region can actually promote the production of Aβ at the level of APP processing. Accordingly, detecting increased levels of these autoantibodies can be an indicator of Alzheimer's disease.

The autoantibodies that are detected according to the present invention are amyloidogenic Aβ₁₋₁₇ antibodies. The term “amyloidogenic Aβ₁₋₁₇ antibody” refers to an antibody that 1) binds to a region of a beta amyloid (Aβ) polypeptide including all or a portion of amino acids 1-17 and 2) increases APP amyloidogenic processing. An increase in APP amyloidogenic processing can be indicated by 1) an increase in a sAPP-β as compared to a control, 2) a decrease in a sAPP-α as compared to a control, and/or 3) an increase in a β-CTF as compared to a control. Accordingly, the present disclosure includes a method for diagnosing Alzheimer's disease in a subject comprising detecting an increase in amyloidogenic Aβ₁₋₁₇ antibody in the subject, or a sample obtained from a subject, as compared to a control, wherein the amyloidogenic Aβ₁₋₁₇ antibody binds to a region of a beta amyloid (Aβ) polypeptide including all or a portion of amino acids 1-17 and wherein the amyloidogenic Aβ₁₋₁₇ antibody increases sAPP-β in an APP cleavage assay as compared to a control. Also included herein is a method for diagnosing Alzheimer's disease in a subject comprising detecting an increase in a sAPP-β polypeptide in a subject as compared to a control. In some embodiments, the increase in sAPP-β is approximately 10%, 20%, 30%, 40%, 50%, or 100% as compared to the control.

Also included herein is a method for diagnosing Alzheimer's disease in a subject comprising detecting an increase in an amyloidogenic Aβ₁₋₁₇ antibody in the subject, or a sample obtained from a subject, as compared to a control, wherein the amyloidogenic Aβ₁₋₁₇ antibody binds to a region of a beta amyloid (Aβ) polypeptide including all or a portion of amino acids 1-17 and wherein the amyloidogenic Aβ₁₋₁₇ antibody decreases sAPP-α in an APP cleavage assay as compared to a control. Also included herein is a method for diagnosing Alzheimer's disease in a subject comprising detecting an decrease in a sAPP-α polypeptide in a subject as compared to a control. In some embodiments, the decrease in sAPP-α is approximately 10%, 20%, 30%, 40%, 50%, or 100% as compared to the control.

Further included herein is a method for diagnosing Alzheimer's disease in a subject comprising detecting an increase in an amyloidogenic Aβ₁₋₁₇ antibody in the subject, or a sample obtained from a subject, as compared to a control wherein the amyloidogenic Aβ₁₋₁₇ antibody binds to a region of a beta amyloid (Aβ) polypeptide including all or a portion of amino acids 1-17 and wherein the amyloidogenic Aβ₁₋₁₇ antibody increases a β-CTF in an APP cleavage assay as compared to a control. Also included herein is a method for diagnosing Alzheimer's disease in a subject comprising detecting an increase in a β-CTF polypeptide in a subject as compared to a control. In some embodiments, the increase in a β-CTF is approximately 10%, 20%, 30%, 40%, 50%, or 100% as compared to the control. The control can be 13-actin in some embodiments and the increase in β-CTF can be expressed as a ratio of β-CTF to β-actin.

The present also disclosure includes a method for diagnosing an APP-related disease in a subject comprising detecting an increase in amyloidogenic Aβ₁₋₁₇ antibody in the subject, or a sample obtained from a subject, as compared to a control, wherein the amyloidogenic Aβ₁₋₁₇ antibody binds to a region of a beta amyloid (Aβ) polypeptide including all or a portion of amino acids 1-17 and wherein the amyloidogenic Aβ₁₋₁₇ antibody increases sAPP-β in an APP cleavage assay as compared to a control. APP-related diseases include, but are not limited to, Alzheimer's disease, autism, Down's syndrome, and traumatic brain injury. Also included herein is a method for diagnosing APP-related disease in a subject comprising detecting an increase in a sAPP-β polypeptide in a subject as compared to a control. In some embodiments, the increase in sAPP-β is approximately 10%, 20%, 30%, 40%, 50%, or 100% as compared to the control.

Also included herein is a method for diagnosing an APP-related disease in a subject comprising detecting an increase in an amyloidogenic Aβ₁₋₁₇ antibody in the subject, or a sample obtained from a subject, as compared to a control, wherein the amyloidogenic Aβ₁₋₁₇ antibody binds to a region of a beta amyloid (Aβ) polypeptide including all or a portion of amino acids 1-17 and wherein the amyloidogenic Aβ₁₋₁₇ antibody decreases sAPP-α in an APP cleavage assay as compared to a control. Also included herein is a method for diagnosing an APP-related disease in a subject comprising detecting an decrease in a sAPP-α polypeptide in a subject as compared to a control. In some embodiments, the decrease in sAPP-α is approximately 10%, 20%, 30%, 40%, 50%, or 100% as compared to the control.

Still further included herein is a method for diagnosing an APP-related disease in a subject comprising detecting an increase in an amyloidogenic Aβ₁₋₁₇ antibody in the subject, or a sample obtained from a subject, as compared to a control wherein the amyloidogenic Aβ₁₋₁₇ antibody binds to a region of a beta amyloid (Aβ) polypeptide including all or a portion of amino acids 1-17 and wherein the amyloidogenic Aβ₁₋₁₇ antibody increases a β-CTF in an APP cleavage assay as compared to a control. Also included herein is a method for diagnosing an APP-related disease in a subject comprising detecting an increase in a β-CTF polypeptide in a subject as compared to a control. In some embodiments, the increase in a β-CTF is approximately 10%, 20%, 30%, 40%, 50%, or 100% as compared to the control. The control can be β-actin in some embodiments and the increase in β-CTF can be expressed as a ratio of β-CTF to β-actin.

An APP cleavage assay can be any assay that detects one or more cleavage products of APP (or APP fragments) including, but not limited to, sAPP-β, sAPP-α, β-CTF, α-CTF and Aβ. Various APP cleavage assays are well-known to those of ordinary skill in the art. In one embodiment, the APP cleavage assay makes use of CHO/APPswe/PS1wt cells and in some further embodiments, the APP cleavage assay makes use of CHO/APPswe/PS1wt cells as described in the Example below. Each APP cleavage product can be identified by commercially available antibodies including, but not limited to, mouse monoclonal 6E10 (human Aβ residues 1-17; Covance, Emeryville, Calif., USA), 4G8 (Aβ residues 17-24; Covance), 1E11 (Aβ residues 1-8; Covance), VPB-203 (Aβ residues 8-17; Vector Laboratories, Burlingame, Calif., USA), 9F1 (Aβ residues 32-40; Calbiochem, La Jolla, Calif., USA), AB10 (human Aβ residues 1-17; Merck Millipore, Billerica, Mass., USA), and Aβ₁₋₁₂ antibody (BAM10, Sigma-Aldrich, St Louis, Mo., USA).

In some embodiments, the amyloidogenic Aβ₁₋₁₇ antibodies, sAPP-β, sAPP-α, β-CTF, α-CTF, and/or Aβ are detected in a sample obtained from a subject. The sample can be a fluid, tissue or other sample. A fluid sample includes, but is not limited to, a sample of urine, blood, semen, sweat, amniotic fluid, cerebrospinal fluid, synovial fluid, pleural fluid, pericardial fluid, and peritoneal fluid. In one embodiment, the sample is a blood sample. In another embodiment, the sample is a cerebrospinal fluid sample. In some embodiments, the sample is a brain tissue sample.

Also provided herein is a method for prognosing an Alzheimer's disease, or an APP-related disease, in a subject comprising detecting an increase or a decrease in an amyloidogenic Aβ₁₋₁₇ antibody in the subject as compared to a control, wherein an increase indicates a worse prognosis and a decrease indicates a more favorable prognosis. Accordingly, the present disclosure includes a method for prognosing Alzheimer's disease, or an APP-related disease, in a subject comprising detecting an increase or a decrease in an amyloidogenic Aβ₁₋₁₇ antibody in the subject as compared to a control, wherein an increase indicates a worse prognosis and a decrease indicates a more favorable prognosis, and wherein the amyloidogenic Aβ₁₋₁₇ antibody binds to a region of a beta amyloid (Aβ) polypeptide including all or a portion of amino acids 1-17, and the amyloidogenic Aβ₁₋₁₇ antibody increases sAPP-β in an APP cleavage assay as compared to a control. Also included herein is a method for prognosing Alzheimer's disease, or an APP-related disease, in a subject, comprising detecting an increase or a decrease in a sAPP-βpolypeptide as compared to a control, wherein an increase indicates a worse prognosis and a decrease indicates a more favorable prognosis. In some embodiments, the increase or decrease in sAPP-β is approximately 10%, 20%, 30%, 40%, 50%, or 100% as compared to the control.

Further included herein is a method for prognosing Alzheimer's disease, or an APP-related disease, in a subject comprising detecting an increase or a decrease in an amyloidogenic Aβ₁₋₁₇ antibody in the subject as compared to a control, wherein an increase indicates a worse prognosis and a decrease indicates a more favorable prognosis, wherein the amyloidogenic Aβ₁₋₁₇ antibody binds to a region of a beta amyloid (Aβ) polypeptide including all or a portion of amino acids 1-17, and wherein the amyloidogenic Aβ₁₋₁₇ antibody decreases sAPP-α in an APP cleavage assay as compared to a control. Also included herein is a method for prognosing Alzheimer's disease, or an APP-related disease, in a subject, comprising detecting an increase or a decrease in a sAPP-α polypeptide as compared to a control, wherein a decrease indicates a worse prognosis and an increase indicates a more favorable prognosis. In some embodiments, the increase or decrease in sAPP-α is approximately 10%, 20%, 30%, 40%, 50%, or 100% as compared to the control.

Further included herein is a method for prognosing Alzheimer's disease, or an APP-related disease, in a subject comprising detecting an increase or a decrease in an amyloidogenic Aβ₁₋₁₇ antibody in the subject as compared to a control, wherein an increase indicates a worse prognosis and a decrease indicates a more favorable prognosis, wherein the amyloidogenic Aβ₁₋₁₇ antibody binds to a region of a beta amyloid (Aβ) polypeptide including all or a portion of amino acids 1-17 and wherein the amyloidogenic Aβ₁₋₁₇ antibody increases a β-CTF in an APP cleavage assay as compared to a control. Also included herein is a method for prognosing Alzheimer's disease, or an APP-related disease, in a subject, comprising detecting an increase or a decrease in a β-CTF polypeptide as compared to a control, wherein an increase indicates a worse prognosis and a decrease indicates a more favorable prognosis. In some embodiments, the increase or decrease in β-CTF is approximately 10%, 20%, 30%, 40%, 50%, or 100% as compared to the control.

Further provided herein is a method for testing the efficacy of an Alzheimer's disease, or an APP-related disease, treatment in a subject comprising detecting a decrease in an amyloidogenic Aβ₁₋₁₇ antibody in the subject as compared to prior to the treatment. Accordingly, the present disclosure includes a method for testing the efficacy of an Alzheimer's disease, or an APP-related disease, treatment in a subject comprising detecting a decrease in an amyloidogenic Aβ₁₋₁₇ antibody in the subject as compared to prior to the treatment, wherein the amyloidogenic Aβ₁₋₁₇ antibody binds to a region of a beta amyloid (Aβ) polypeptide including all or a portion of amino acids 1-17 and wherein the amyloidogenic Aβ₁₋₁₇ antibody increases sAPP-βpolypeptide in an APP cleavage assay as compared to a control. Also included herein is a method for testing the efficacy of an Alzheimer's disease, or an APP-related disease, treatment in a subject comprising detecting an decrease in a sAPP-βpolypeptide in a subject as compared to prior to the treatment. In some embodiments, the decrease in sAPP-β is approximately 10%, 20%, 30%, 40%, 50%, or 100% as compared to the control.

Also included herein is a method for testing the efficacy of an Alzheimer's disease, or an APP-related disease, treatment in a subject comprising detecting a decrease in an amyloidogenic Aβ₁₋₁₇ antibody in the subject as compared to prior to the treatment, wherein the amyloidogenic Aβ₁₋₁₇ antibody binds to a region of a beta amyloid (Aβ) polypeptide including all or a portion of amino acids 1-17 and wherein the amyloidogenic Aβ₁₋₁₇ antibody decreases sAPP-α polypeptide in an APP cleavage assay as compared to a control. Also included herein is a method for testing the efficacy of an Alzheimer's disease, or an APP-related disease, treatment in a subject comprising detecting an increase in a sAPP-α polypeptide in a subject as compared to prior to the treatment. In some embodiments, the increase in sAPP-α is approximately 10%, 20%, 30%, 40%, 50%, or 100% as compared to the control.

Further included herein is a method for testing the efficacy of an Alzheimer's disease, or an APP-related disease, treatment in a subject comprising detecting a decrease in an amyloidogenic Aβ₁₋₁₇ antibody in the subject as compared to prior to the treatment wherein the amyloidogenic Aβ₁₋₁₇ antibody binds to a region of a beta amyloid (Aβ) polypeptide including all or a portion of amino acids 1-17 and wherein the amyloidogenic Aβ₁₋₁₇ antibody increases a β-CTF polypeptide in an APP cleavage assay as compared to a prior to the treatment. Also included herein is a method for testing the efficacy of an Alzheimer's disease, or an APP-related disease, treatment in a subject comprising detecting a decrease in a β-CTF polypeptide in a subject as compared to prior to the treatment. In some embodiments, the decrease in β-CTF is approximately 10%, 20%, 30%, 40%, 50%, or 100% as compared to the control.

Furthermore, future vaccine strategies may need to take into account antibody binding in the Aβ₁₋₁₇ region of APP as targeting this region may impart deleterious effects in the form of amyloidogenic APP processing. Targeting this region may also dilute the Aβ-clearing effects of these autoantibodies.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims. All patents, patent applications, and publications referenced herein are incorporated by reference in their entirety for all purposes.

### EXAMPLES

### Example 1

### Aβ₁₋₁₇ Autoantibodies from AD Patients Promote β-Secretase APP Cleavage

CHO/APPswe/PS1wt cells were treated with sera-derived auto-Aβ₁₋₁₇ antibodies from AD patients (n=10) and non-demented controls (n=10). Neither group contained individuals with a known co-morbid autoimmune disease. An increase in Aβ species in the cells treated with concentrated total auto-Aβ₁₋₁₇ antibodies from AD patients was observed as compared to age-matched controls. Likewise, there was a corresponding increase in the ratio of β-C-terminal fragment (β-CTF) to β-actin in this same group as determined by immunoblot analysis of the cell lysates (FIG. 1).

FIG. 1 shows that the concentrated Aβ autoantibodies from AD patients promote β-secretase cleavage of APP in CHO/APPswe/PS1wt cells. The concentrated sera were individually prepared from AD patients and normal aging controls (Ctrl). FIG. 1a shows autoantibodies against Aβ peptide 1-17 were measured in the concentrated sera by ELISA. Data are presented as mean (±SD) in a dot-plot (anti-Aβ₁₋₁₇ IgG mg/mL) from 10 Alzheimer's disease patients and 10 age-matched controls. A t-test did not reveal a significant difference between Alzheimer's disease and normal aging controls in terms of quantity of auto-Aβ₁₋₁₇ antibodies (p>0.05).

For functional assessment of APP processing, CHO/APPswe/PS1wt cells were treated with AD or normal age-matched control-derived concentrated auto-Aβ₁₋₁₇ antibodies at 1.25 μg/mL for 3 hours. The top panel of FIG. 1b shows that Aβ species were analyzed in conditioned media from the CHO/APPswe/PS1wt cells by immunoblot (IB) analysis using Aβ₁₋₁₇ antibody (6E10). The second panel of FIG. 1b shows that human IgG heavy chain (IgGH) and IgG light (IgGL) were analyzed by IB as the internal reference using an anti-human IgG antibody (anti-human Aβ). The third panel of FIG. 1b shows that cell lysates were prepared and subjected to IB analysis of APP CTFs pAb751/770 (C-APP). The fourth panel of FIG. 1b shows that the β-CTF band was further confirmed by the IB using 6E10 following blot striping. As indicated below this panel, an anti-β-actin antibody was used an internal reference control for the third and fourth panels of FIG. 1b. FIG. 1c shows a bar graph representing a densitometry analysis showing the ratio of Aβ to sAPP-α (one of the light exposed blots) (top panel) or β-CTF to β-actin. Aβ and β-CTF IB results are representative of results obtained for 10 cases per group. A t-test revealed a significant difference between AD cases and normal aging controls (n=10) in either ratio of Aβ to sAPP-α or β-CTF to β-actin. **p<0.01.

### Patients

All samples were obtained from ProteoGenex Inc. (Culver City, Calif., USA). Ten patients (5 males and 5 females) with probable Alzheimer's disease diagnosed according to DSM-IV criteria (MMSE, mean 16.6±2 SD) were included in the study if they were 60-80 years old (mean 75.7±5 SD) and did not have a diagnosis of comorbid autoimmune disease. Healthy controls were matched with AD patients (n=10) solely on the basis of age (mean 65.6±2.1 SD) and gender. Sample collection from clinical sites in Moscow, Russia, were approved by an independent ethics committee in accordance with Russian law, US federal law (HIPPA), WHO, ICH, and GCP guidelines. All participating patients gave written informed consent.

### Concentration of Human Serum

Human sera were concentrated under vacuum at ambient temperature (25° C.). Auto-Ab₁₋₁₇ antibody levels in the concentrated sera were measured by ELISA. Briefly, 96-well ELISA plates were coated with 100 μL Aβ₁₋₁₇ (1 μg/mL) and incubated overnight at 4° C. Plates were washed 5 times with washing buffer and then blocked for 1 hour at 37° C. Following blocking, the plates were washed 4 times with washing buffer and the concentrated human serum samples were applied (100 μL/well) in duplicate or triplicate and incubated at 4° C. overnight. The plates were then washed 3 times with washing buffer and anti-Human IgG was diluted 1:10,000 and incubated for 1 hour. After incubation, the plates were washed 3 times and developed with tetramethylbenzidine substrate-chromogen (Dako, Carpinteria, Calif., USA). The reaction was stopped with 2 N sulfuric acid (50 μL) and the plates were analyzed spectrophotometrically at 450 nm.

### Antibodies

Several well-characterized Aβ antibodies were used: mouse monoclonal 6E10 (human Aβ residues 1-17; Covance, Emeryville, Calif., USA), 4G8 (Aβ residues 17-24; Covance), 1E11 (Aβ residues 1-8; Covance), VPB-203 (Aβ residues 8-17; Vector Laboratories, Burlingame, Calif., USA), 9F1 (Aβ residues 32-40; Calbiochem, La Jolla, Calif., USA), AB10 (human Aβ residues 1-17; Merck Millipore, Billerica, Mass., USA), and Aβ₁₋₁₂ antibody (BAM10, Sigma-Aldrich, St Louis, Mo., USA). Mouse IgG1 and IgG2b (Biolegend, La Jolla, Calif., USA) were used as controls. Medium was changed to provide fresh medium to cells just prior to each treatment. Final Aβ antibody concentrations in each treatment were 0.63, 1.25, and 2.5 μg/mL. Cells were incubated with individual antibodies for 3 hours.

### Cell Lines and Cell Culture

Chinese hamster ovary (CHO) cell lines and human neuroblastoma SH-SY5Y cells, both with stable coexpression of human APP bearing the Swedish mutation (APPswe) and wild-type human PSEN1 (PS1wt), were engineered as previously described [Weggen, S., Eriksen, J. L., Sagi, S. A. et al. (2003) Journal of Biological Chemistry 278, 30748-30754; Hahn, S., Brüning, T. et al. (2011) Journal of Neurochemistry 116(3), 385-395]. CHO/APPswe/PS1wt cells were maintained in Dulbecco's modified Eagle's medium with 10% fetal bovine serum, 1 mM sodium pyruvate and 100 units/mL penicillin/streptomycin (Invitrogen, Carlsbad, Calif.). SH/APPswe/PS1wt cells were cultured in complete Dulbecco's modified Eagle's medium/F12 medium supplemented with 10% fetal bovine serum, 1% geneticin (G418; 40 mg/mL, Invitrogen) and hygromycin (50 mg/mL, Invitrogen). Cells were plated in 24-well plates at a density of 1-105 cells per well. After overnight incubation, the cells were treated with Aβ-antibodies at dosages of 0.63, 1.25, and 2.5 μg/mL for 3 hours.

### Mice

All mice were housed and maintained in the College of Medicine Animal Facility at the University of South Florida (USF), and all experiments were conducted in compliance with protocols approved by the USF Institutional Animal Care and Use Committee. Double transgenic ‘Swedish’ mutant APPK595N/M596L (APPswe)+PS1DE9 B6C3-Tg 85 Dbo/J strain (PSAPP mice), 8-month-old mice were purchased from the Jackson Laboratory (Bar Harbor, Me., USA). Because sex differences can impact Aβ deposition [Jankowsky, J. L., Slunt, H. H., Ratovitski, T. et al. (2001) Biomolecular Engineering 17(6), 157-165], only females were used in the analyses (n=3).

### Immunoblot Analysis

Supernatants of the cells were collected and Aβ monomers and oligomers were visualized using immunoblot protocol. Cultured cells were lysed in ice-cold lysis buffer as described previously [Tan, J., Town, T., Crawford, F. et al. (2002) Nature Neuroscience 5, 1288-1293]. All antibodies were diluted in Tris-buffered saline (TBS) containing 5% (w/v) non-fat dry milk. Blots were developed using the Luminol reagent (Thermo Fisher Scientific, Waltham, Mass., USA). Densitometric analysis was performed as described previously [Rezai-Zadeh, K., Shytle, D., Sun, N. et al. (2005) Journal of Neuroscience 25, 8807-8814] using a FluorS Multiimager with Quantity One software (Bio-Rad, Hercules, Calif., USA). Antibodies used for immunoblot analysis included rabbit anti-APP C-terminus polyclonal antibody (pAb369, 1:1000) provided by Dr. Sam Gandy, rabbit anti-APP C-terminus polyclonal antibody (pAb751/770, 1:1000, Calbiochem), N-terminal Aβ 6E10 (1:1000; Covance), and β-actin (1:1500; as an internal reference control; Sigma-Aldrich).

### Example 2

### Antibody Against N-Terminal Region of Aβ Markedly Increases Aβ Production

An in vitro system was used to examine the effects of Aβ antibodies raised against various regions of Aβ on APP processing. CHO/APPswe/PS1wt cells were treated with antibodies raised against Aβ's N-terminal residues: 1-8, 8-17, 1-17, 17-26, or against the C-terminal residues (33-42) of Aβ, at 1.25 μg/mL (based on the upper limit for Aβ₁₋₁₇ concentration in AD patient serum yielding amyloidogenic processing in vitro; FIG. 1) for 3 hours. There were significant differences between Aβ₁₋₁₇ antibody (6E10) and other antibodies when compared to control IgG1 and Aβ₃₃₋₄₂ as demonstrated by immunoblot for Aβ species (FIG. 2a).

Furthermore, immunoblot analysis of cell lysates for β-CTF revealed significantly greater β-CTF generation in cells treated with 6E10 compared with control IgG1 and Aβ₃₃₋₄₂ antibody (FIG. 2b). This β-CTF was further confirmed by the IB using BAM10 (FIG. 2c). In addition, similar results were also observed in SH/APPswe/PS1wt cells treated with Aβ antibody against Aβ₁₋₁₇ peptide (6E10) (FIGS. 2d and 2e). Finally, antibody binding to the cell membranes of CHO/APPswe/PS1wt cells after 1 hour incubation was examined by confocal microscopy. Higher binding of anti-Aβ₁₋₁₇ antibody (6E10) was detected as compared to control isotype IgG1 on these cell membranes (FIGS. 2f and 2g). In addition, SH/APPswe/PS1wt cells were used for this binding assay and similar results were observed in SH/APPswe/PS1wt cells stained with fluorescent-dye conjugated 6E10 (data not shown).

### Example 3

### Aβ₁₋₁₇ Antibody Dose-Dependently Promotes Aβ Production

To determine the dose-response relationship, treated CHO/APPswe/PS1wt cells were treated with Aβ₁₋₁₇ antibody (6E10) at various concentrations as indicated for 3 hours. Significant differences in Aβ40 levels were found between 6E10 at 2.5 μg/mL and 1.25 or 0.63 μg/mL by ELISA and immunoblot analyses of the cell supernatants (FIGS. 3a and 3b). As expected, there was also a significant dose-dependent increase in β-CTF (FIG. 3c). In addition, Aβ₁₇₋₂₆ antibody (4G8) was used at similar concentrations for 3 hours and results similar to Aβ₁₋₁₇ antibody (6E10) were obtained (FIGS. 3d-3f).

More specifically, FIG. 3 shows CHO/APPswe/PS1wt cells were treated with 6E10 at various concentrations as indicated for 3 hours. Supernatants were collected and subjected to Aβ ELISA (a) and IB (b) analyses using BAM10. Cell lysates were prepared and subjected to IB analysis (c) for APP processing by pAb751/770 (C-APP). In addition, the β-CTF band was further confirmed by the IB using BAM10 (data not shown). For panel (a), secreted Aβ peptide species were analyzed by ELISA. Aβ levels are presented as relative fold mean (±SD) over IgG1 control. The results are representative of three independent experiments with n=3 for each condition. A t-test revealed significant differences in Aβ levels between Ab₁₋₁₇ antibody at 2.5 μg/mL and 1.25 or 0.63 μg/mL. For panels (d-f), in parallel, Aβ₁₇₋₂₆ antibody (4G8) was used at the same concentrations for 3 hours. Results similar to Aβ₁₋₁₇ antibody (6E10) were observed. For panel (d), secreted Aβ 40, 42 peptides were analyzed by ELISA antibody. Densitometry analysis shows the ratios of Aβ to sAPP-α (b, e), β-CTF to β-actin (c, f) as indicated below the figures. ***p<0.001.

### ELISA

To measure Aβ levels with 4G8 and IgG2b antibody treatment, Aβ_{40,42} ELISA kits (Invitrogen) were used following the manufacturer's instructions with modifications. In treatment groups not utilizing N-terminal Aβ antibodies, the manufacturer's instructions were strictly followed. In treatment groups utilizing N-terminal Aβ antibodies, to avoid interference with N-terminal capture antibodies, 96-well ELISA plates were coated with 100 μL Aβ₃₂₋₄₀ (1 mg/mL) in phosphate-buffered saline (PBS) and incubated overnight at 4° C. Plates were washed 5 times with washing buffer (0.05% Tween-20 in PBS) and then blocked (300 μL/well) for 1 hour at 37° C. with 1% bovine serum albumin+0.05% Tween-20 in PBS. Following blocking, the plates were washed 4 times with washing buffer and the samples were applied (100 μL/well) in duplicate or triplicate and incubated at 4° C. overnight. The plates were then washed 3 times with washing buffer and 6E10 (2 μg/mL) was added for detection of Aβ. Following another wash, goat anti-mouse IgG with horseradish peroxidase conjugation was diluted 1:2000 and incubated for 30 minutes. After incubation, the plates were washed 3 times, developed with tetramethylbenzidine substrate-chromogen (Dako). The reaction was stopped with 2 N sulfuric acid (50 μL) and the plates were analyzed spectrophotometrically at 450 nm.

### Statistical Analysis

All data were normally distributed; therefore, in instances of single mean comparisons, Levene's test for equality of variances followed by the t-test for independent samples were used to assess significance. In instances of multiple mean comparisons, one-way analysis of variance (ANOVA) was used. Alpha was set at 0.05 for all analyses. The statistical package for the social sciences release IBM SPSS 18.0 (IBM, Armonk, N.Y., USA) was used for all data analyses.

### Example 4

### Aβ₁₋₁₇ Antibody Dampens α-Secretase Activity

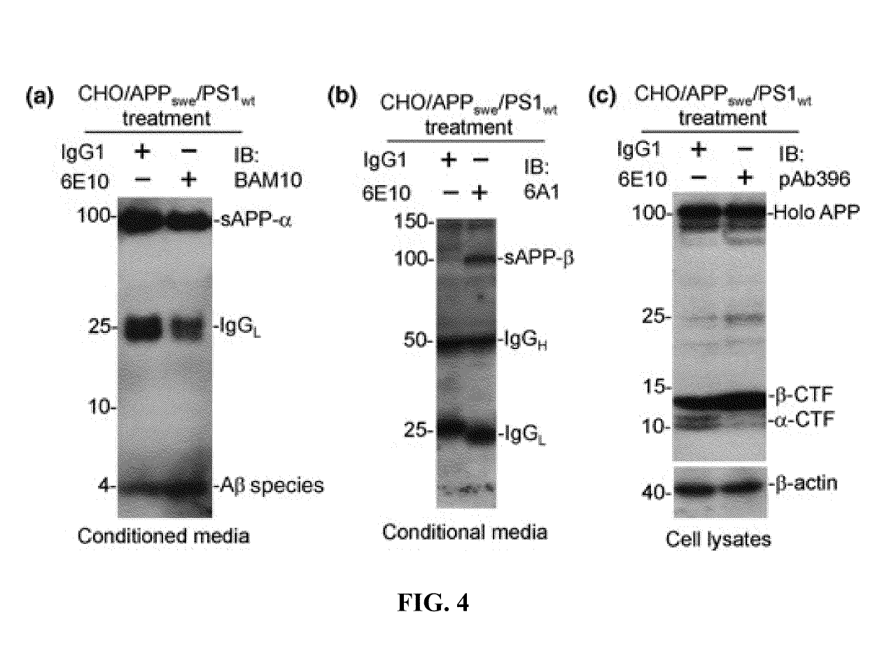

To determine how the Aβ₁₋₁₇ antibody may promote Aβ production, CHO/APPswe/PS1wt cells were treated with Aβ₁₋₁₇ antibody (6E10), under the same conditions as above, for immunoblot analysis of APP metabolites: Aβ, sAPP-α, and sAPP-β. Upon application of anti-N-terminal Aβ₁₋₁₇ antibody (6E10), a significant decrease in sAPP-α, corresponding with an increase in Aβ in conditioned media was found (FIG. 4a). Furthermore, there was a relative increase in sAPP-β in conditioned media from the Aβ₁₋₁₇ antibody (6E10) treated cells compared with controls by immunoblot analysis (FIG. 4b). Finally, cells exposed to the Aβ₁₋₁₇ antibody (6E10) displayed a higher ratio of β- to α-CTF in the cell lysate by immunoblot analysis (FIG. 4c).

More specifically, FIG. 4 shows CHO/APPswe/PS1wt cells were treated with Aβ₁₋₁₇ antibody (6E10) or IgG1 isotype control at 1.25 μg/mL for 3 hours. Conditioned media were collected and subjected to immunoblot analysis for Aβ species, sAPP-α (a) and sAPP-β (b). For panel (a), IB analysis using an anti-Aβ₁₋₁₂ monoclonal antibody (BAM10) shows secreted sAPP-α and Aβ species. Mouse IgG light (IgGL) was also shown by the IB as indicated. For panel (b), IB analysis using antibody specifically against soluble APP-β of Swedish type cleaved by β-secretase (6A1) shows secreted sAPP-β. Cell lysates were prepared and subjected to IB analysis for APP processing (c). For panel (c), IB analysis using anti-C-terminal APP rabbit antibody 369 (pAb369) shows full-length holo APP and two bands corresponding to β-CTF (C99) and α-CTF (C83). These results are representative of three independent experiments with n=3 for each condition.

### Example 5

### Aβ₁₋₁₇ Antibody Promotes Amyloidogenic APP Processing In Vivo

PSAPP mice at 8 months of age were subjected to intracerebroventricular (i.c.v.) injection with Aβ₁₋₁₇ antibody (6E10), Aβ₃₃₋₄₂ antibody, or IgG1 control at 5 μg/mouse. Animals were anesthetized using isoflurane (chamber induction at 4-5% isoflurane, intubation and maintenance at 1-2%). After reflexes were checked to ensure that mice were unconscious, they were positioned on a stereotaxic instrument (Stoelting Lab Standard, Wood Dale, Ill., USA). The Aβ antibody (6E10) and isotype control IgG1 were dissolved in sterile distilled water at a concentration of 1 μg/lL. Aβ antibody and control IgG1 (5 μL) were injected into the left lateral ventricle with a microsyringe at a rate 1 μL/min with the following coordinates relative to bregma: −0.6 mm anterior/posterior, +1.2 mm medial/lateral, and −3.0 mm dorsal/ventral, per previous methods [Giunta, B., Obregon, D., Hou, H. et al. (2006) Brain Research 1123, 216-225]. The needle was left in place for 5 minutes after injection before being withdrawn. At 24 and 48 hours after the i.c.v. injections, animals were killed with isofluorane and brain tissues were collected. All dissected brain tissues were rapidly frozen for immunoblot analysis.

As shown in FIG. 5, the immunoblot analysis of brain homogenates using a monoclonal anti-Aβ₁₋₁₂ antibody (BAM10) indicated that Aβspecies were increased (FIG. 5a) in the 6E10 treated group compared to the Aβ₃₃₋₄₂ antibody or IgG1 control groups. Correspondingly, the ratio of β- to α-CTF in this group was significantly higher than the other Aβ₃₃₋₄₂ antibody or IgG1 control groups by immunoblot analysis (FIG. 5b).

More specifically, FIG. 5 shows PSAPP mice at 8 months of age were intracerebroventricular (i.c.v.) injected with Aβ₁₋₁₇ antibody (6E10), Aβ₃₃₋₄₂ antibody (9F1) or control IgG1 at 5 μg/mouse and euthanized 24 and 48 hours after the treatment. Mouse brain homogenates were prepared (the right half of brain tissues (the non-injection side)) and subjected to IB analysis for APP processing. For panel (a), IB analysis using Aβ₁₋₁₂ antibody (BAM10) shows total APP and Aβ species. For panel (b), IB analysis using pAb369 shows full-length holo APP and two bands corresponding to β-CTF (C99) and α-CTF (C83). Densitometry analysis shows the ratios of Aβ to β-actin (a) and β-CTF to β-actin (b) as indicated below the figures. IB data presented here are representative of results obtained for 3 female mice per group at each time point.

1. A method for diagnosing an Alzheimer's disease in a subject comprising detecting an increase in an amyloidogenic Aβ₁₋₁₇ antibody in the subject as compared to a control.

2. The method of claim 1, wherein the increase in amyloidogenic Aβ₁₋₁₇ antibody is indicated by detecting an increase in a sAPP-β polypeptide.

3. The method of claim 2, wherein the sAPP-β polypeptide is detected in a sample obtained from the subject.

4. The method of claim 2, wherein a sample is obtained from the subject, an amyloid precursor protein (APP) cleavage assay is performed with the sample, and the sAPP-β polypeptide is detected as a product of the cleavage assay.

5. The method of claim 1, wherein the increase in amyloidogenic Aβ₁₋₁₇ antibody is indicated by detecting a decrease in a sAPP-α polypeptide.

6. The method of claim 5, wherein the sAPP-α polypeptide is detected in a sample obtained from the subject.

7. The method of claim 5, wherein a sample is obtained from the subject, an amyloid precursor protein (APP) cleavage assay is performed with the sample, and the sAPP-α polypeptide is detected as a product of the cleavage assay.

8. The method of claim 1, wherein the increase in amyloidogenic Aβ₁₋₁₇ antibody is indicated by detecting an increase in a β-CTF polypeptide.

9. The method of claim 8, wherein the β-CTF polypeptide is detected in a sample obtained from the subject.

10. The method of claim 8, wherein a sample is obtained from the subject, an amyloid precursor protein (APP) cleavage assay is performed with the sample, and the β-CTF polypeptide is detected as a product of the cleavage assay.

11. The method of claim 1, wherein the amyloidogenic Aβ₁₋₁₇ antibody is obtained from a blood sample.

12. The method of claim 1, wherein a control sample is obtained from a subject not having Alzheimer's disease symptoms.

13. A method for testing efficacy of an Alzheimer's disease treatment in a subject comprising detecting a decrease in an amyloidogenic Aβ₁₋₁₇ antibody in the subject as compared to before treatment of the subject.

14. The method of claim 13, wherein the decrease in amyloidogenic Aβ₁₋₁₇ antibody is indicated by detecting a decrease in sAPP-β polypeptide.

15. The method of claim 14, wherein the sAPP-β polypeptide is detected in a sample obtained from the subject.

16. The method of claim 14, wherein a sample is obtained from the subject, an amyloid precursor protein (APP) cleavage assay is performed with the sample, and the sAPP-β polypeptide is detected as a product of the cleavage assay.

17. The method of claim 13, wherein the decrease in amyloidogenic Aβ₁₋₁₇ antibody is indicated by detecting an increase in sAPP-α polypeptide.

18. The method of claim 17, wherein the sAPP-α polypeptide is detected in a sample obtained from the subject.

19. The method of claim 13, wherein the decrease in amyloidogenic Aβ₁₋₁₇ antibody is indicated by detecting a decrease in a β-CTF polypeptide.

20. The method of claim 19, wherein the β-CTF polypeptide is detected in a sample obtained from the subject.

21. The method of claim 13, wherein the amyloidogenic Aβ₁₋₁₇ antibody is obtained from a blood sample.
